(19) United States
(12) Patent Application Publication
(10) Pub. No.: US 2025/0290886 A1
(43) Pub. Date: Sep. 18, 2025

(54) GAS SENSOR PACKAGE STRUCTURE

(71) Applicant: WEN-LO HSIEH, Kaohsiung City (TW)

(72) Inventor: WEN-LO HSIEH, Kaohsiung City (TW)

(21) Appl. No.: 18/602,047

(22) Filed: Mar. 12, 2024

(51) Int. Cl. G01N 27/12; G01N 33/00

(52) U.S. Cl. CPC G01N 27/125; G01N 33/0009

(57) ABSTRACT

The present invention includes a substrate, a cover body, a sensing chip, and a breathable film formed of polytetrafluoroethylene (PTFE). The breathable film is embedded under a top wall of the cover body to selectively allow outside air to enter an internal space of the cover body through a through hole. The breathable film includes an upper surface, a lower surface, and a riveting hole, where the upper surface and the lower surface include a plurality of vent holes, the breathable film is placed in a mold before the cover body is formed by the mold, so that an infused epoxy molding compound can be filled into the vent holes and the riveting hole of the breathable film. After curing, the breathable film and the cover body are integrated into one body.

### TECHNICAL FIELD

The present invention relates to a gas sensor package structure, and in particular, to a gas sensor package structure. In the structure, when a cover body is formed by a mold, a breathable film is pre-placed in the mold, and after molding, the breathable film and the cover body are integrated into one body, to effectively prevent the breathable film from detaching.

### RELATED ART

The Industrial Technology Research Institute discloses an invention filed with U.S. Pat. No. 1,290,358 of the People's Republic of China and entitled “GAS SENSOR PACKAGE STRUCTURE AND MANUFACTURING METHOD THEREFOR”. The present invention is to fix at least one micro-sensing chip in an accommodating space of a gas sensor package structure carrier, and to electrically connect the micro-sensing chip to the carrier. A filter member is fixed on a top surface of the accommodating space of the carrier, and the filter member includes at least a structural mesh and a film located on an outer surface of the structural mesh. The filter member includes an upper (first) structural mesh and a lower (second) structural mesh made of metal, plastic, or ceramics. A powder filler including activated carbon, porous ceramics, or metal oxides is pressed between the upper and lower structural meshes. The second structural mesh includes a mesh hole formed by mechanical processing or chemical etching and is designed to prevent the filler from being exposed. A film is formed on an outer surface of the second structural mesh by pasting, screen printing, vapor deposition, or in another manner. The film is a polymer material and is a waterproof and breathable PTFE (polytetrafluoroethylene, or referred to as Teflon) microporous film. The filter member can isolate light, water vapor, and contaminants in an environment, thereby overcoming a shortcoming of susceptibility to contamination in conventional technologies. This patent is the first patent document in patent exploration to disclose use of a PTFE film as a barrier to water vapor and contaminants in an environment.

The US 2019/0204281A1 patent discloses a gas sensor structure. The gas sensor package includes a package substrate, a controller on the package substrate, a plurality of gas sensors on the controller, and a cover body on the package substrate. The cover body includes holes extending between a first surface and a second surface of the cover body, the first surface of the cover body faces away from the package substrate, and the second surface of the cover body faces the package substrate. The gas sensor senses different types of gases.

This patent applies for a gas sensor package structure. The sensor includes a substrate with wires and contacts thereon, more than one chip is bonded to the substrate, and the wires connect chip pads to substrate pads. The plurality of chips may be placed side by side or stacked upwards, and the wires may all be connected to the substrate pads or may be connected to each other from top to bottom. A sealing cover body may be plastic packaging or plastic cover sealing. The cover body needs to leave holes, and a cavity needs to be reserved above the sensing chip, so that the chip can be exposed to gas to produce a sensing function. A piece of material referred to as a breathable film is applied to an inside or outside of a sealant or the cover body through an adhesive film referred to as adhesive Film or plugged into vent holes of the cover body. The cover body may be metal or plastic.

The patent claims a variety of structures within the scope of the patent, but a careful inspection of all structures reveals that the breathable film is a main adhesive material used to bond the breathable film to the sealant or the cover body. However, a structure shown in the patent application diagram is similar to a structure disclosed by a well-known market research and product analysis unit in which a PTFE film of a gas sensor may be easily “peeled off without any residue” from a plastic cover body of the package. This is judged as an application example of this patent.

### SUMMARY

A main objective of the present invention is to provide a gas sensor to effectively prevent a breathable film from detaching and resolve a problem that a polytetrafluoroethylene (PTFE) breathable film of a conventional gas sensor is not easily combined with a cover body made of another material. The gas sensor has high efficiency, low costs, and extended service life, and can be used in gas sensor package structures in various harsh environments and medical fields.

To achieve the foregoing objective, the present invention discloses a gas sensor package structure, including a substrate, a cover body, at least one sensing chip, and a breathable film formed of polytetrafluoroethylene (PTFE), where the substrate includes an upper surface, and at least one first solder point is arranged on the upper surface. The cover body is made of thermosetting epoxy through die-casting or injection molding, where the cover body includes an opening toward the substrate and a top wall away from the substrate, a ring wall surrounds a periphery of the cover body and is connected to the top wall, the top wall and a peripheral wall define an internal space, at least one through hole in communication with the internal space is provided on the top wall, and the ring wall of the cover body is combined with the upper surface of the substrate. The sensing chip is located in the internal space of the cover body and combined with the upper surface of the substrate, where the sensing chip is configured to sense at least one gas in the internal space, the sensing chip includes at least one second solder point, and the second solder point is electrically connected to the first solder point of the substrate through a wire. The breathable film formed of polytetrafluoroethylene (PTFE) is embedded under the top wall of the cover body and includes an upper surface, a lower surface, and at least one riveting hole penetrating the upper surface and the lower surface, where the breathable film may selectively allow partial external air to enter the internal space of the cover body through the through hole, the upper surface and the lower surface of the breathable film are rough surfaces and include a plurality of vent holes, when the cover body is formed, the breathable film is pre-placed in a mold, and when the epoxy is infused to form the cover body, the thermosetting epoxy is die-cast or injection molded to fill and penetrate into the vent holes on the upper surface and the lower surface of the breathable film and fill the riveting hole of the breathable film, so that the breathable film is embedded into the cover body after curing, and the breathable film and the cover body are integrated into one body.

When the cover body is formed by the mold, the thermosetting epoxy at a fixed flow rate is poured into the mold through injection or die-casting for molding, and is pressurized to penetrate into the upper surface and the lower surface of the breathable film, and a penetration thickness accounts for 30% to 50% of a thickness of the breathable film.

The thermosetting epoxy forming the cover body penetrates into the vent holes of the breathable film to a depth of 10 μm to 16 μm.

When the thermosetting epoxy forming the cover body reaches a curing condition and completes an infusing action, the thermosetting epoxy is pressurized to penetrate into the vent holes of the breathable film; and when the thermosetting epoxy reaches the curing condition, the breathable film and the cover body are integrated into one body.

Two stripe-shaped through holes parallel to each other or a plurality of circular through holes are provided on the top wall of the cover body.

The cover body and the breathable film are in one of square, circular, or polygonal shapes, and the riveting holes are respectively located at a junction of a side wall of the breathable film and the cover body.

Therefore, the present invention may resolve a problem that a polytetrafluoroethylene (PTFE) breathable film of a conventional gas sensor is not easily combined with a cover body made of another material. The gas sensor has high efficiency, low costs, and extended service life, and can be used in gas sensor package structures in various harsh environments and medical fields.

### BRIEF DESCRIPTION OF THE DRAWINGS

FIG. 1 is a top view according to an embodiment of the present invention;

FIG. 2 is a cross-sectional view according to an embodiment of the present invention;

FIG. 3 is a front view of a cover body and a breathable film in circular shapes according to the present invention;

FIG. 4 is a top view of a cover body and a breathable film in circular shapes according to the present invention;

FIG. 5 is a front view of a cover body and a breathable film in polygonal shapes according to the present invention; and

FIG. 6 is a top view of a cover body and a breathable film in polygonal shapes according to the present invention.

### DETAILED DESCRIPTION

Embodiments of the present invention are described in detail below with reference to the accompanying drawings, the accompanying drawings are mainly simplified schematic diagrams, and only exemplify the basic structure of the present invention schematically. Therefore, only components related to the present invention are shown in the drawings, and are not drawn according to a quantity, shapes, and size ratios of the components during actual implementation. During actual implementation, specifications and sizes of the components are selective designs, and component layouts may be more complex.

The following description of embodiments is provided to exemplify the specific embodiments for implementation of the present invention with reference to accompanying drawings. The directional terms mentioned in the present invention, for example, “above”, “below”, “front”, “back”, refer to the directions in the appended drawings. Therefore, the used direction terms are intended to describe and understand this application, but are not intended to limit this application. In addition, in the specification, unless explicitly described as contrary, the word “include” is understood as referring to including the element, but does not exclude any other elements.

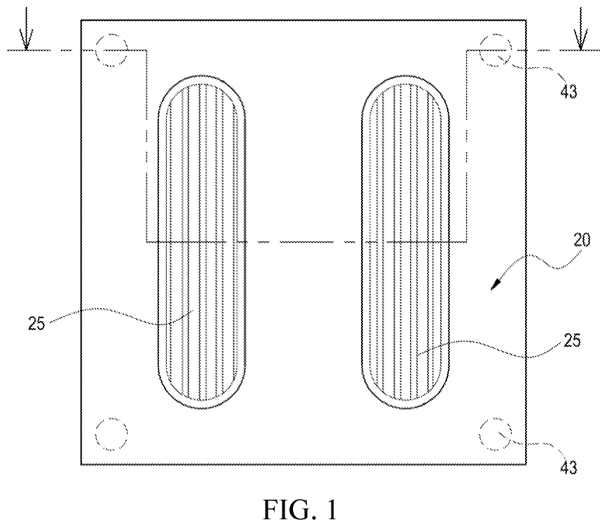

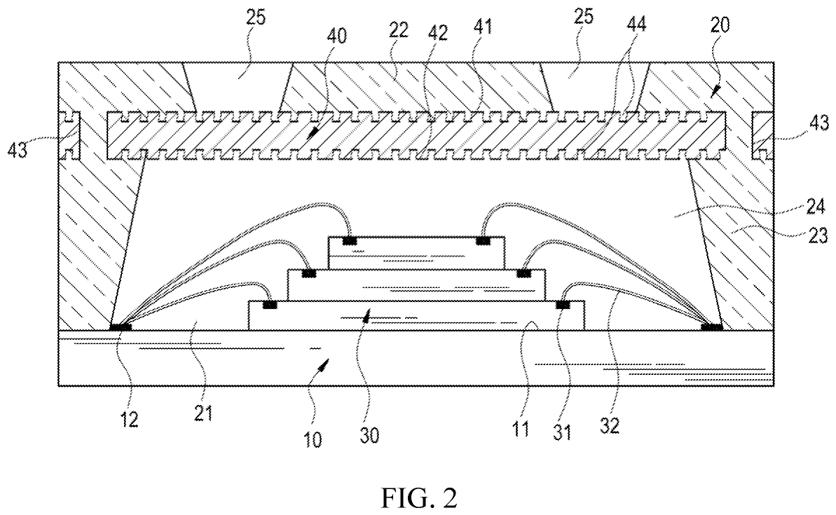

FIG. 1 and FIG. 2 show a preferred embodiment of a gas sensor package structure according to the present invention, where the structure includes a substrate 10, a cover body 20, at least one sensing chip 30, and a breathable film 40 formed of polytetrafluoroethylene (PTFE).

In this embodiment, the substrate 10 may be a printed circuit board or a flexible printed circuit. The substrate 10 includes an upper surface 11, at least one first solder point 12 is arranged on the upper surface 11 of the substrate 10, and the first solder point 12 is electrically connected to an electronic component on the substrate 10.

In this embodiment, the cover body 20 is in a square shape, and is made of thermosetting epoxy through die-casting or injection molding. The cover body 20 may further be selected from a metal material or a plastic material, and includes an opening 21 toward the substrate 10. The cover body 20 includes a top wall 22 in a direction away from the substrate 10, a ring wall 23 surrounds a periphery of the cover body 20 and is connected to the top wall 22, the top wall 22 and the ring wall 23 enable the cover body 20 to define an internal space 24, at least one through hole 25 is provided on the top wall 22, and the through hole 25 runs through the top wall 22 and is in communication with the internal space 24 of the cover body 20. In this embodiment, two strip-shaped through holes 25 parallel to each other are provided on the top wall 22, or the through hole 25 may be in a circular shape or in another shape, and the cover body 20 is formed by a mold for molding. The ring wall 23 of the cover body 20 adjacent to the substrate 10 is combined with the upper surface 11 of the substrate 10.

At least one sensing chip 30 is located in the internal space 24 of the cover body 20 and is combined with the upper surface 11 of the substrate 10. The sensing chip 30 is configured to sense at least one gas in the internal space 24 of the cover body 20. In this embodiment, the sensing chips 30 may selectively use, for example, at least one of a hydrogen sulfide sensing chip, a carbon monoxide sensing chip, a carbon dioxide sensing chip, a combustible gas sensing chip, a volatile organic gas sensing chip, a nitrogen dioxide sensing chip, an ammonia sensing chip, and a chlorine dioxide sensing chip, and are respectively configured to sense gases such as hydrogen sulfide, carbon monoxide, carbon dioxide, a flammable gas, a volatile organic gas, nitrogen dioxide, ammonia, and chlorine dioxide. The sensing chip 30 includes at least one second solder point 31, and the second solder point 31 is electrically connected to the first solder point 12 of the substrate 10 through a wire 32. The wire 32 may be a metal wire with high conductivity such as gold, silver, or copper. When there is a plurality of sensing chips 30, the plurality of sensing chips 30 is stacked on each other and bonded to the upper surface 11 of the substrate 10 through epoxy or another viscous polymer adhesive.

The breathable film 40 includes an upper surface 41, a lower surface 42, and at least one riveting hole 43 penetrating the upper surface 41 and the lower surface 42, where the riveting hole 43 is formed by laser drilling. In addition to retaining the unique physical/chemical properties of original polytetrafluoroethylene (PTFE), the breathable film 40 is also porous and may take into account the properties of waterproof, dustproof, breathable, anti-fouling, and resistant to strong acids and alkalis. The breathable film 40 is in a square shape relative to the cover body 20, and is embedded and combined under the top wall 22 of the cover body 20, and the breathable film 40 may selectively allow partial external gas to enter the internal space 24 of the cover body 20 through the through hole 25. In this embodiment, two riveting holes 43 (a quantity and the shape of the riveting holes 43 are not limited) penetrating the upper surface 41 and the lower surface 42 are respectively provided at the junction between the breathable film 40 and the two side walls of the cover body 20, and the upper surface 41 and the lower surface 42 of the breathable film 40 are rough surfaces, and each includes a plurality of vent holes 44. When the cover body 20 is formed, the breathable film 40 is pre-placed in the mold, so that when the epoxy molding compound is infused to form the cover body 20, the epoxy molding compound used to form the cover body 20 may be filled into the vent holes 44 on the upper surface 41 and the lower surface 42 of the breathable film 40, and filled into the riveting holes 43 of the breathable film 40, so that the breathable film 40 is embedded into the cover body 20 after curing, and the breathable film 40 and the cover body 20 are integrated into one body, as shown in FIG. 2.

In this embodiment, when the cover body 20 is formed by a mold, epoxy at a fixed flow rate is poured into the mold and pressurized to penetrate into a thickness of the upper surface 41 and the lower surface 42 of the breathable film 40 accounting for 30% to 50% of a thickness of the breathable film 40. When a temperature of the epoxy molding compound is at 40° C., the epoxy molding compound may be pressurized to penetrate into the vent holes 44 of the breathable film 40. After a temperature of the thermosetting epoxy drops to 20° C., curing is achieved, so that the breathable film 40 and the cover body 20 are integrated into one body. The thermosetting epoxy penetrates into the vent holes 44 of the breathable film 40 to a depth of 10 μm to 16 μm.

The main features of the present invention lie in that the breathable film 40 is selected from polytetrafluoroethylene (PTFE) fibers with the vent holes 44 in a vertical direction. The upper surface 41 and the lower surface 42 of the breathable film 40 each include a plurality of vent holes 44 fibers in the vertical direction, and a plurality of riveting holes 43 is provided on the breathable film 40. When the cover body 20 is molded, the breathable film 40 is pre-placed in the mold. When the epoxy molding compound is poured into the mold to form the cover body 20, the epoxy molding compound may be filled into the riveting holes 43 and the vent holes 44 of the breathable film 40, the breathable film 40 is embedded into the cover body 20 after curing, and the breathable film 40 and the cover body 20 are integrated into one body. Therefore, the breathable film 40 has an advantage of not being detaching from the cover body 20, to effectively prevent the breathable film 40 from detaching from the gas sensor. The gas sensor has high efficiency, low costs, and extended service life, and can be used in package structures in various harsh environments and medical fields, and effectively resolves a problem that the polytetrafluoroethylene (PTFE) breathable film of the conventional gas sensor is not easily combined with the cover body made of another material.

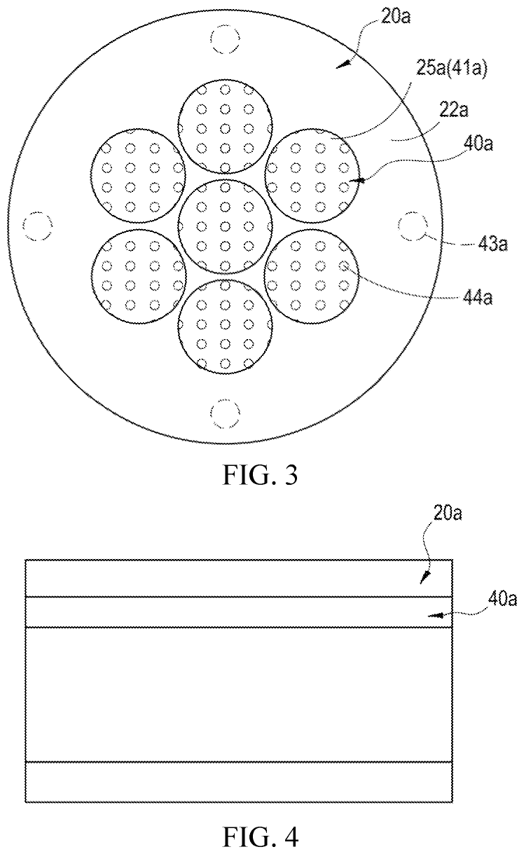

During implementation and application, the cover body 20a and the breathable film 40a may be in circular shapes. As shown in FIG. 3 and FIG. 4, a plurality of circular through holes 25a is provided on the top wall 22a of the cover body 20a. The upper surface 41a and the lower surface (not shown in the figure) of the breathable film 40a each include a plurality of vent holes 44a fibers in the vertical direction, and four riveting holes 43a penetrating the upper surface 41a and the lower surface are provided on an outer periphery adjacent to the breathable film 40a.

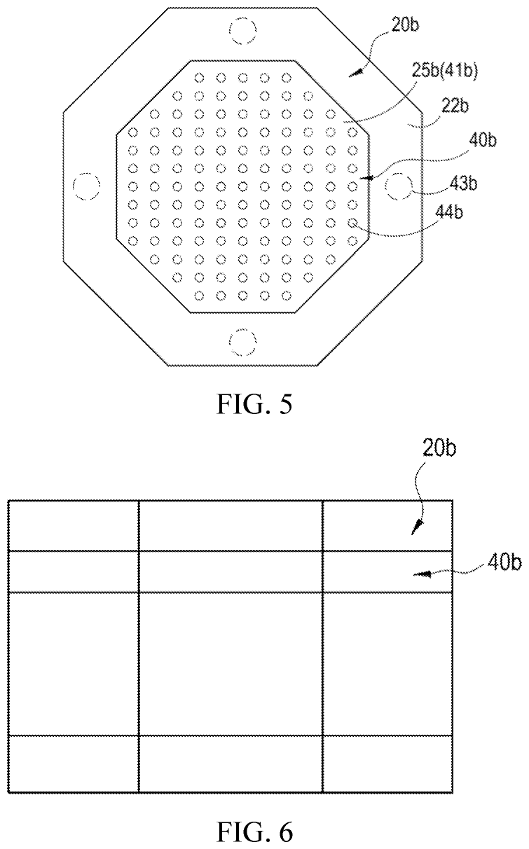

During implementation and application, the cover body 20b and the breathable film 40b may be in polygonal shapes. The octagon shown in FIG. 5 and FIG. 6, a polygonal through hole 25b is provided on the top wall 22b of the cover body 20b. The upper surface 41b and the lower surface (not shown in the figure) of the breathable film 40b each include a plurality of vent holes 44b fibers in the vertical direction, and four riveting holes 43b penetrating the upper surface 41b and the lower surface are provided on an outer periphery adjacent to the breathable film 40b.

The present invention has been described with exemplary embodiments above. However, one skilled in the art should understand that the embodiments are only used to describe the present invention, and are not intended to limit the scope of the present invention. It should be noted that, any changes and replacements equivalent to the embodiments shall fall within the scope of the present invention. Therefore, the protection scope of the present invention shall be subject to the scope of the following claims.

What is claimed is:

1. A gas sensor package structure, comprising:
a substrate, comprising an upper surface, wherein at least one first solder point is arranged on the upper surface;
a cover body, made of thermosetting epoxy through die-casting or injection molding, wherein the cover body comprises an opening toward the substrate and a top wall away from the substrate, a ring wall surrounds a periphery of the cover body and is connected to the top wall, the top wall and a peripheral wall define an internal space, at least one through hole in communication with the internal space is provided on the top wall, and the ring wall of the cover body is combined with the upper surface of the substrate;
at least one sensing chip, located in the internal space of the cover body and combined with the upper surface of the substrate, wherein the sensing chip is configured to sense at least one gas in the internal space, the sensing chip comprises at least one second solder point, and the second solder point is electrically connected to the first solder point of the substrate through a wire; and
a breathable film formed of polytetrafluoroethylene (PTFE), embedded under the top wall of the cover body and comprising an upper surface, a lower surface, and at least one riveting hole penetrating the upper surface and the lower surface, wherein the breathable film selectively allows partial external air to enter the internal space of the cover body through the through hole, the upper surface and the lower surface of the breathable film are rough surfaces and comprise a plurality of vent holes, when the cover body is formed, the breathable film is pre-placed in a mold, and when the epoxy is infused to form the cover body, the thermosetting epoxy is die-cast or injection molded to fill and penetrate into the vent holes on the upper surface and the lower surface of the breathable film and fill the riveting hole of the breathable film, so that the breathable film is embedded into the cover body after curing, and the breathable film and the cover body are integrated into one body.

2. The gas sensor package structure according to claim 1, wherein when the cover body is formed by the mold, the thermosetting epoxy at a fixed flow rate is poured into the mold through injection or die-casting for molding, and is pressurized to penetrate into the upper surface and the lower surface of the breathable film, and a penetration thickness accounts for 30% to 50% of a thickness of the breathable film.

3. The gas sensor package structure according to claim 1, wherein the thermosetting epoxy forming the cover body penetrates into the vent holes of the breathable film to a depth of 10 μm to 16 μm.

4. The gas sensor package structure according to claim 1, wherein when the thermosetting epoxy forming the cover body reaches a curing condition and completes an infusing action, the thermosetting epoxy is pressurized to penetrate into the vent holes of the breathable film; and when the thermosetting epoxy reaches the curing condition, the breathable film and the cover body are integrated into one body.

5. The gas sensor package structure according to claim 1, wherein two strip-shaped through holes parallel to each other are provided on the top wall of the cover body.

6. The gas sensor package structure according to claim 1, wherein a plurality of circular through holes is provided on the top wall of the cover body.

7. The gas sensor package structure according to claim 1, wherein the cover body and the breathable film are in one of square, circular, or polygonal shapes, and the riveting holes are respectively located at a junction of two side walls of the breathable film and the cover body.
